(19) United States
(12) Patent Application Publication
(10) Pub. No.: US 2008/0039631 A1
(43) Pub. Date: Feb. 14, 2008

(54) Process For Preparing N-(Substituted Arylmethyl) -4-Substituted-4-(Disubstituted Methyl) Piperidines And Intermediates

(75) Inventors: Jaidev Goudar, West Windsor, NJ (US); David S. Rosen, Princeton, NJ (US); Walter H. Yeager, Yardley, PA (US); Guozhi Wang, Freshmeadow, NJ (US); Qun Zhang, Princeton, NJ (US); Russell Patera, Hamilton, NJ (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Crop Science AG, Monheim (DE)

(21) Appl. No.: 11/662,598

(22) PCT Filed: Sep. 12, 2005
(86) PCT No.: PCT/US05/32279

§ 371 Date: Jul. 18, 2007

Related U.S. Application Data
(60) Provisional application No. 60/609,533, filed on Sep. 13, 2004.

(51) Int. Cl. C07D 401/12; C07C 37/00; C07C 41/01; C07D 211/52

(52) U.S. Cl. 546/194; 546/216; 568/626; 568/809

(57) ABSTRACT

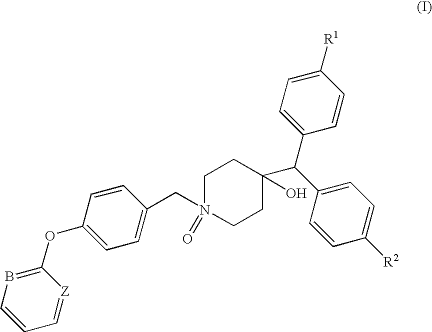

An improved process is described for preparing a N-(substituted arylmethyl)-4-substituted-4-(disubstituted methyl)piperidine of formula (I): wherein R¹ and R² are independently selected from the group consisting of halogen, CF₃, OCF₃, OCHF₂, OCF₂CHF₂ and SF₅; and Z and B are independently selected from the group consisting of CH and N; as well as processes for preparing intermediates useful in the overall process.

This application claims the benefit of U.S. Provisional Application No. 60/609,533, filed Sep. 13, 2004.

### FIELD OF THE INVENTION

This invention is in the field of chemical processes; more specifically, an improved process for preparing N-(substituted arylmethyl)-4-substituted-4-(disubstituted methyl)piperidines, processes for preparing intermediates useful in this process and novel intermediates useful in this process.

### BACKGROUND

N-(substituted arylmethyl)-4-substituted-4-(disubstituted methyl)piperidines are useful insecticides and have been described in PCT published application WO 2005/036961 the disclosure of which is incorporated herein by reference. Disadvantages of processes disclosed in WO 2005/036961 to produce these compounds include less than optimal yields, highly exothermic reactions due to the presence of fluorine, less than optimal cycle times and high catalyst loadings. The present invention improves yield, cycle times and catalyst loading and reduces the exothermic nature of certain of the reactions involved in producing N-(substituted arylmethyl)-4-substituted-4-(disubstituted methyl)piperidines.

### SUMMARY OF THE INVENTION

The present invention is directed to (a) an overall process for preparing N-(substituted arylmethyl)-4-substituted-4-(disubstituted methyl)piperidines of formula I:

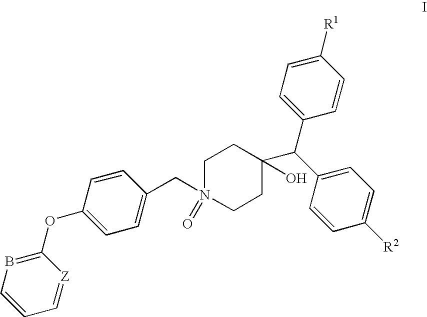

wherein
R¹ and R² are independently selected from the group consisting of halogen, CF₃, OCF₃, OCHF₂, OCF₂CHF₂ and SF₅; and
Z and B are independently selected from the group consisting of CH and N;

(b) individual steps involved in the process, and (c) novel intermediates.

The overall process comprises the following eight steps:

a) forming intermediate (B) : 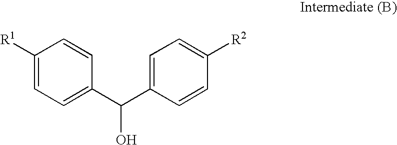
wherein R¹ and R² are independently selected from the group consisting of halogen, CF₃, OCF₃, OCHF₂, OCF₂CHF₂ and SF₅; by reacting two substituted aryl halides of formula (A-1) and formula (A-2):

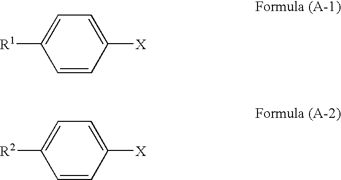

wherein
X is halogen; and
R¹ and R² are as defined above and can be the same or different;

with a Grignard reagent and an alkyl formate;

b) halogenating intermediate (B) to form intermediate (C):

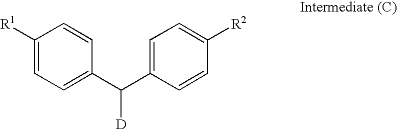

wherein
D is halogen; and
R¹ and R² are as defined above;

c) reacting intermediate (C) with a compound of formula (D):

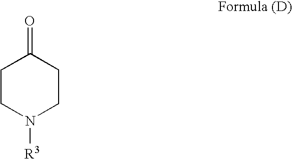

wherein R³ is selected from the group consisting of hydrogen, benzyl, substituted benzyl, t-butoxycarbonyl and trimethylsilyl; to form intermediate (E):

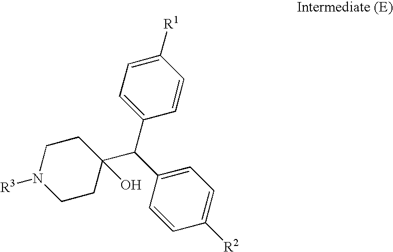

wherein R¹, R² and R³ are as defined above;

d) reacting intermediate (E) with an acid in the presence of a catalyst to form intermediate (F):

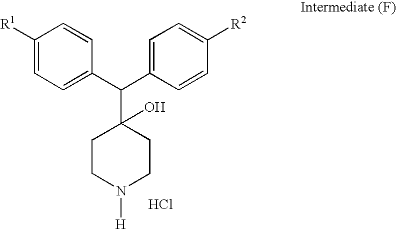

wherein R¹ and R² are as defined above;

e) reacting a substituted phenol and a compound selected from the group consisting of 2-halopyridine, 2-halopyrimidine and halobenzene to form intermediate (G):

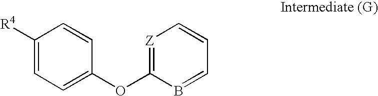

wherein
R⁴ is —CH₃ or —CHO; and
Z and B are as defined above;

f) when R⁴ in intermediate (G) is —CH₃, halogenating intermediate (G) to form intermediate (H):

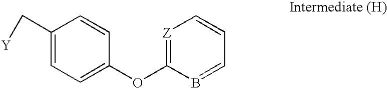

wherein
Y is halogen; and
Z and B are as defined above;

g) reacting intermediate (F) and a compound selected from the group consisting of i) intermediate (G) wherein R⁴ is —CHO and ii) intermediate (H) to form intermediate (J):

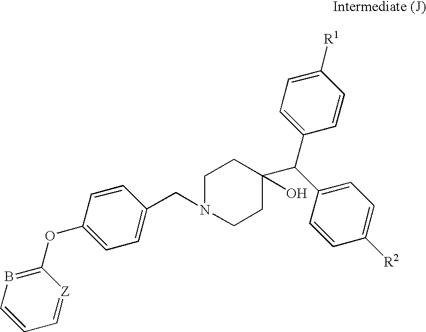

wherein R¹, R², Z and B are as defined above; and

h) oxidizing intermediate (J) to form a compound of formula I.

Steps a, c and g, as well as the compound identified as formula E, are also part of the invention.

### DETAILED DESCRIPTION OF THE INVENTION

The present invention relates to an overall process, individual steps of the process and novel intermediates involved in preparing a N-(substituted arylmethyl)-4-substituted-4-(disubstituted methyl)piperidine of formula I:

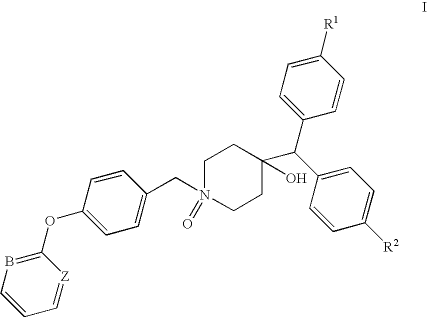

wherein
R¹ and R² are independently selected from the group consisting of halogen, CF₃, OCF₃, OCHF₂, OCF₂CHF₂ and SF₅; and
Z and B are independently selected from the group consisting of CH and N.

The overall process comprises the following eight steps:

a) forming intermediate (B): 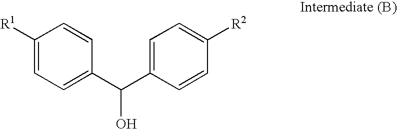
wherein R¹ and R² are independently selected from the group consisting of halogen, CF₃, OCF₃, OCHF₂, OCF₂CHF₂ and SF₅; by reacting two substituted aryl halides of formula (A-1) and formula (A-2):

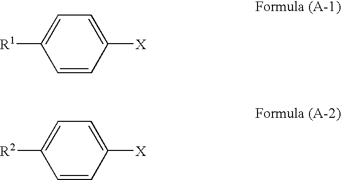

wherein
X is halogen; and
R¹ and R² are as defined above and can be the same or different;

with a Grignard reagent and an alkyl formate;

b) halogenating intermediate (B) to form intermediate (C):

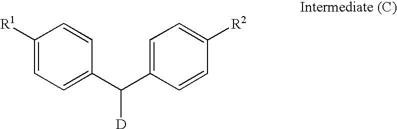

wherein
D is halogen; and
R¹ and R² are as defined above;

c) reacting intermediate (C) with a compound of formula (D):

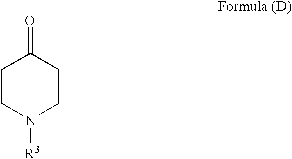

wherein R³ is selected from the group consisting of hydrogen, benzyl, substituted benzyl, t-butoxycarbonyl and trimethylsilyl;

to form intermediate (E):

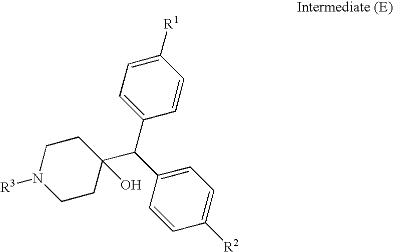

wherein R¹, R² and R³ are as defined above; d) reacting intermediate (E) with an acid in the presence of a catalyst to form intermediate (F):

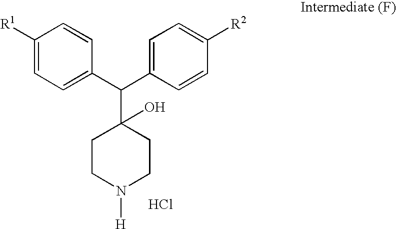

wherein R¹ and R² are as defined above;

e) reacting a substituted phenol and a compound selected from the group consisting of 2-halopyridine, 2-halopyrimidine and halobenzene to form intermediate (G):

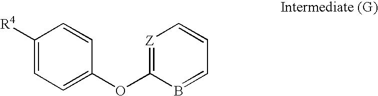

wherein
R⁴ is —CH₃ or —CHO; and
Z and B are as defined above;

f) when R⁴ in intermediate (G) is —CH₃, halogenating intermediate (G) to form intermediate (H):

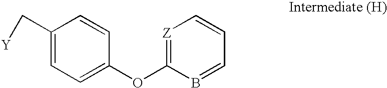

wherein
Y is halogen; and
Z and B are as defined above;

g) reacting intermediate (F) and a compound selected from the group consisting of i) intermediate (G) wherein R⁴ is —CHO and ii) intermediate (H) to form intermediate (J):

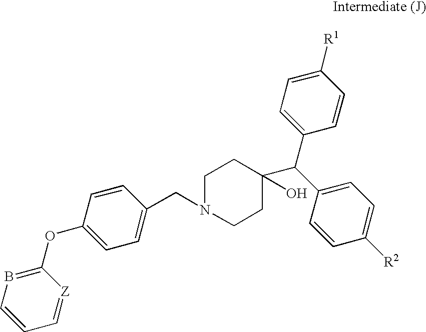

wherein R¹, R², Z and B are as defined above; and

h) oxidizing intermediate (J) to form a compound of formula I.

Preferably, R¹ and R² are independently selected from the group consisting of CF₃, OCF₃ and OCHF₂; X and D are bromine or chlorine; Y is bromine, iodine or chlorine; R³ is benzyl, t-butoxycarbonyl or trimethylsilyl. More preferably, R¹ and R² are OCF₃; X and D are bromine. Also preferred, Z is N and B is CH.

The reacting of step a) can be conducted with ethyl formate or methyl formate as the alkyl formate; the Grignard reagent used in step a) is formed by reacting an alkyl magnesium halide with one of the substituted aryl halides. Preferably, the reacting of step a) is conducted at a temperature in the range of from −20° C. to 30° C.; and the alkyl magnesium halide is i-propyl magnesium chloride or i-propyl magnesium bromide.

The halogenating of step b) can be conducted with hydrogen bromide and acetic acid; in an organic solvent. Preferably, the organic solvent is a hydrocarbon solvent such as heptane, hexane or petroleum ether or an aromatic solvent such as toluene or xylene.

The reacting of step c) can be conducted in the presence of an organometallic reagent. Preferably, the reacting of step c) is conducted at a temperature in the range of from −20° C. to −100° C., more particularly from −50° C. to −100° C.; and the organometallic reagent is t-butyl lithium, sec-butyl lithium or n-butyl lithium.

The reacting of step d) can be conducted with formic acid; with a palladium catalyst; in the presence of alcohol solvents. Preferably, the reacting of step d) is conducted at a temperature in the range of from ambient temperature to 130° C., more particularly from ambient temperature to 100° C.; the alcohol solvents are methanol, ethanol, propanol or butanol; and the palladium catalyst is palladium on a support such as Pd(OH)₂/C, Pd/C, Pd/SiO₂ or Pd/Al₂O₃. More preferably, the alcohol solvent is methanol.

The reacting of step e) can be conducted in the presence of a base and a catalytic amount of copper catalyst. The substituted phenol reacted in step e) is 4-methylphenol or 4-hydroxybenzaldehyde. Preferably, the reacting of step e) is of 4-methylphenol or 4-hydroxybenzaldehyde and 2-chloropyridine; the reacting is conducted at a temperature in the range of from 125° C. to 180° C.; the base is carbonate or hydroxide; and the copper catalyst is copper, copper chloride, copper oxide, copper bromide or copper carbonate.

The reacting of step g) when R⁴ is CHO can be conducted in the presence of sodium borohydride and a solvent. Preferably, the solvent is tetrahydrofuran, dioxane, dichloroethane, dichloromethane or acetonitrile. The reacting of step g) when intermediate (F) and intermediate (H) are reacted can be conducted in the presence of a carbonate, a solvent and optionally a phase transfer catalyst. The solvent can be toluene or methyl isobutyl ketone. The phase transfer catalyst can be polyethylene glycol, dimethylaminopyridine, triethylamine, p-toluenesulfonic acid, phosphorous pentoxide, pyridine or phase transfer catalysts such as quaternary ammonium salts or quaternary phosphonium salts or mixtures thereof.

The oxidizing of step h) can be conducted in the presence of an oxidizing agent and a solvent. Preferably, the oxidizing of step h) is conducted at a temperature in the range of from ambient temperature to 60° C.; the oxidizing agent is peroxide and the solvent is selected from alcohol solvents such as methanol, ethanol, propanol and butanol.

Another embodiment of the present invention is an improved process (step a above) for preparing a compound of formula B:

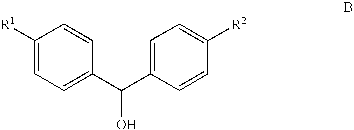

wherein R¹ and R² are independently selected from the group consisting of halogen, CF₃, OCF₃, OCHF₂, OCF₂CHF₂ and SF₅;

said process comprising reacting two substituted aryl halides of formula (A-1) and formula (A-2):

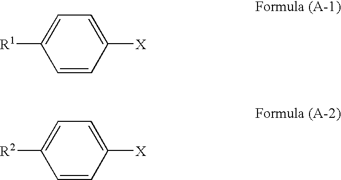

wherein
X is halogen; and
R¹ and R² are as defined above and can be the same or different;

with a Grignard reagent and an alkyl formate.

Another embodiment of the present invention is a process (step c above) for preparing a compound of formula E:

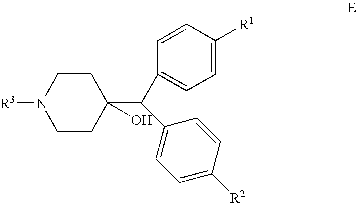

wherein
R¹ and R² are independently selected from the group consisting of halogen, CF₃, OCF₃, OCHF₂, OCF₂CHF₂ and SF₅; and
R³ is selected from the group consisting of hydrogen, benzyl, substituted benzyl, t-butoxycarbonyl and trimethylsilyl;

said process comprising reacting a compound of formula C:

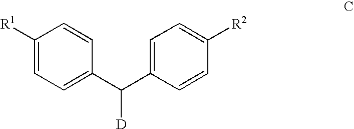

wherein
D is halogen; and
R¹ and R² are as defined above; with a compound of formula (D):

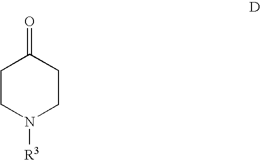

wherein R³ is as defined above.

Another embodiment of the present invention is a process (step g above) for preparing a compound of formula J:

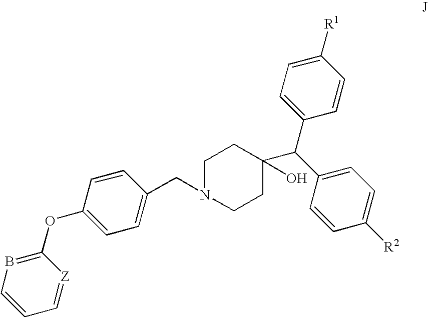

wherein
R¹ and R² are independently selected from the group consisting of halogen, CF₃, OCF₃, OCHF₂, OCF₂CHF₂ and SF₅; and
Z and B are independently selected from the group consisting of CH and N;

said process comprising reacting a compound of formula F:

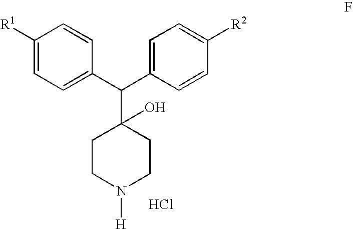

and a compound selected from the group consisting of i) a compound of formula G:

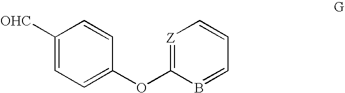

wherein Z and B are as defined above;

and ii) a compound of formula H:

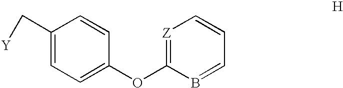

wherein
Y is halogen; and
Z and B are as defined above.

Yet another embodiment of the present invention is a compound of formula E:

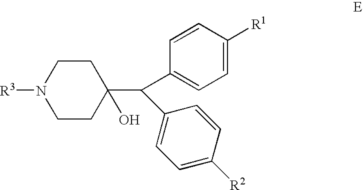

wherein
R¹ and R² are independently selected from the group consisting of halogen, CF₃, OCF₃, OCHF₂, OCF₂CHF₂ and SF₅; and
R³ is selected from the group consisting of benzyl and trimethylsilyl;

which may be prepared by the process described above.

The modifier “about” is used herein to indicate that certain preferred operating ranges, such as ranges for molar ratios for reactants, material amounts, and temperature, are not fixedly determined. The meaning will often be apparent to one of ordinary skill. For example, a recitation of a temperature range of about 120° C to about 135° C. in reference to, for example, an organic chemical reaction would be interpreted to include other like temperatures that can be expected to favor a useful reaction rate for the reaction, such as 105° C. or 150° C. Where guidance from the experience of those of ordinary skill is lacking, guidance from the context is lacking, and where a more specific rule is not recited below, the “about” range shall be not more than 10% of the absolute value of an end point or 10% of the range recited, whichever is less.

As used in this specification and unless otherwise indicated the substituent terms “alkyl”, “alkoxy”, and “haloalkyl”, used alone or as part of a larger moiety, includes straight or branched chains of at least one or two carbon atoms, as appropriate to the substituent, and preferably up to 12 carbon atoms, more preferably up to ten carbon atoms, most preferably up to seven carbon atoms. The term “aryl” refers to phenyl or naphthyl optionally substituted with one or more halogen, alkyl, alkoxy, or haloalkyl. “Halogen”, “halide” or “halo” refers to fluorine, bromine, iodine, or chlorine. The term “ambient temperature” refers to a temperature in the range of about 20° C. to about 30° C. Certain solvents, catalysts, and the like are known by their acronyms. These include the acronyms “DMF” meaning N,N-dimethylformamide and “THF” meaning tetrahydrofuran.

The following examples illustrate the processes of the present invention.

### EXAMPLE 1

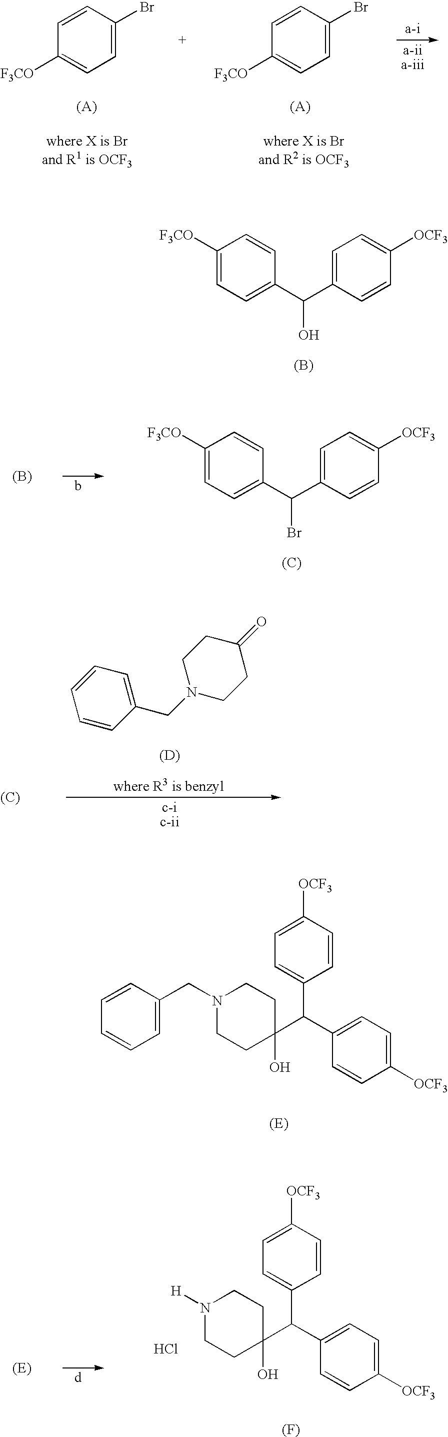

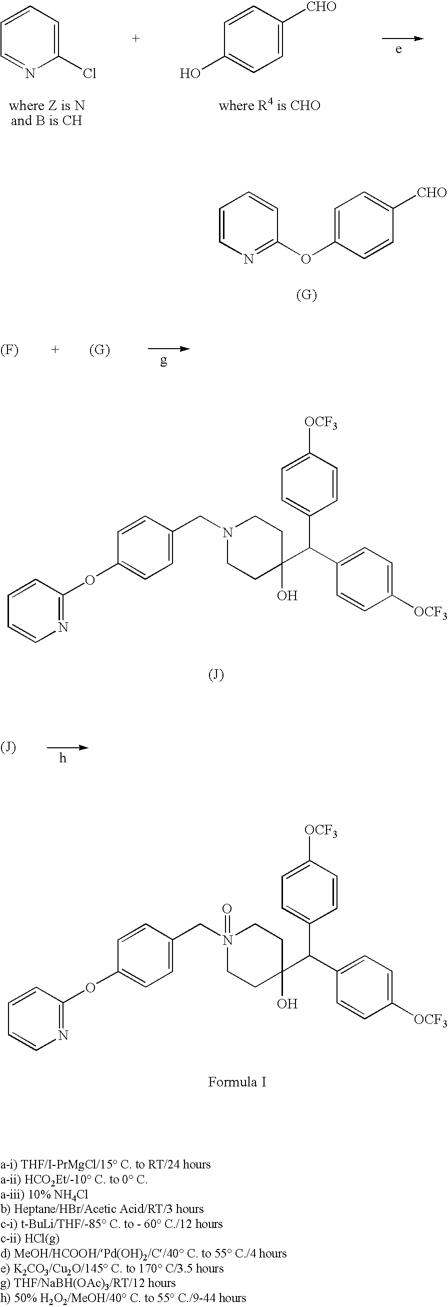

In the first step as depicted in Example 1, two molecules of a substituted aryl halide, for example, the known compound 4-bromo-1-(trifluoromethoxy)benzene (A), were reacted with a Grignard reagent and an alkyl formate, for example, ethyl formate to form bis[4-(trifluoromethoxy)phenyl]methan-1-ol (B). Intermediate (B) was then reacted under acidic conditions with hydrogen bromide, to afford the corresponding (4-{bromo[4-(trifluoromethoxy)phenyl]methyl}phenoxy)trifluoromethane (C). Intermediate (C) was then lithiated, for example with butyl lithium, and then reacted with an appropriately N-substituted piperidin-4-one, formula (D), for example 1-benzylpiperidin-4-one, at a temperature in the range of −85° C. to −60° C. to afford the corresponding 4-{bis[4-(trifluoromethoxy)phenyl]methyl}-1-benzylpiperidin-4-ol (E). Intermediate (E) was then reacted with an acid, for example formic acid, in the presence of a catalyst, for example a palladium catalyst, to form the hydrogen chloride salt of 4-{bis[4-(trifluoromethoxy)phenyl]methyl}piperidin-4-ol (F). Next, an appropriately substituted phenol, for example, the known compound 4-hydroxybenzaldehyde, was reacted with a halopyridine, for example 2-chloropyridine, in the presence of potassium carbonate and a catalytic amount of copper oxide at a temperature in the range of 145° C. to 170° C. to form 4-(2-pyridyloxy)benzaldehyde (G). Intermediate (F) was then reacted with Intermediate (G) in the presence of sodium triacetoxyborohydride to form 4-{bis[4-(trifluoromethoxy)phenyl]methyl}-1-[4-(2-pyridyloxy)phenyl)methyl]piperidin-4-ol (J). Intermediate (J) was then oxidized with hydrogen peroxide at a temperature in the range of 40° C. to 55° C. to form 4-{bis[4-(trifluoromethoxy)phenyl]methyl}-4-hydroxy-1-[(4-(2-pyridyloxy)phenyl)methyl]piperidin-1-one (Formula I).

### EXAMPLE 2

Steps a) through d) are the same as in Example 1 to form (F)

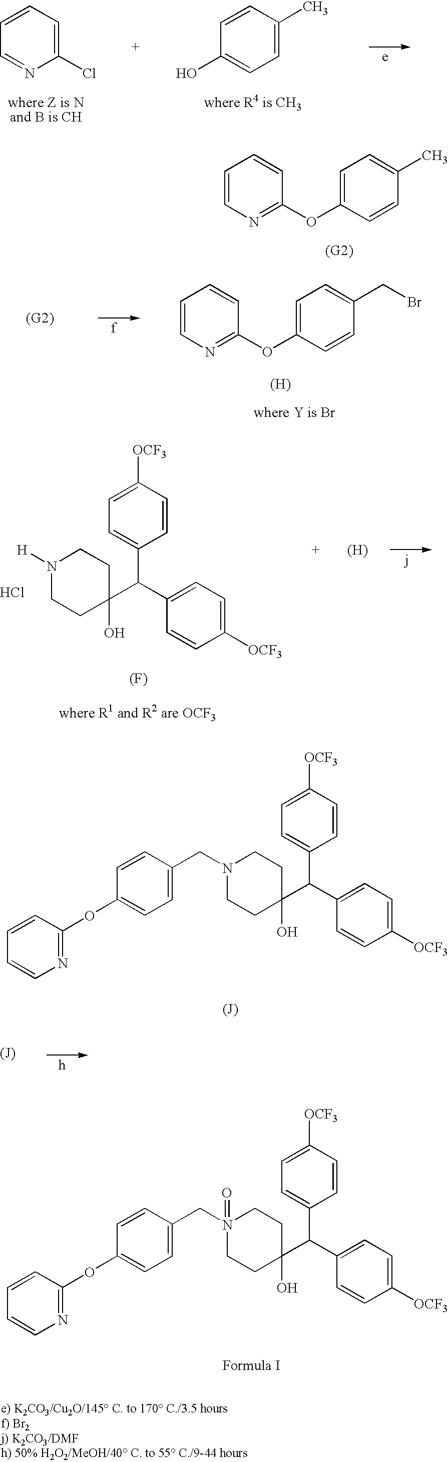

In the first step of Example 2, an appropriately substituted phenol, for example, the known compound 4-methyl phenol, can be reacted with a halopyridine, for example 2-chloropyridine, in the presence of potassium carbonate and a catalytic amount of copper oxide at a temperature in the range of 145° C. to 170° C. to form 2-(4-methylphenoxy)pyridine (G2). Intermediate (G2) can then be halogenated with, for example bromine, to form 2-[4-(bromomethyl)phenoxy]pyridine (H).

Intermediate (F), made as in Example 1, can then be reacted with Intermediate (H) in the presence of potassium carbonate to form 4-{bis[4-(trifluoromethoxy)phenyl]methyl}-1-[(4-(2-pyridyloxy)phenyl)methyl]piperidin-4-ol (J). Intermediate (J) can then be oxidized as in Example 1 to form 4-{bis[4-(trifluoromethoxy)phenyl]methyl}-4-hydroxy-1-[(4-(2-pyridyloxy)phenyl)methyl]piperidin-1-one (Formula I).

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly this invention includes all modifications encompassed within the spirit and scope as defined by the following claims.

What is claimed is:

1. A process for preparing an N-(substituted arylmethyl)-4-substituted-4-(disubstituted methyl)piperidine of formula I: 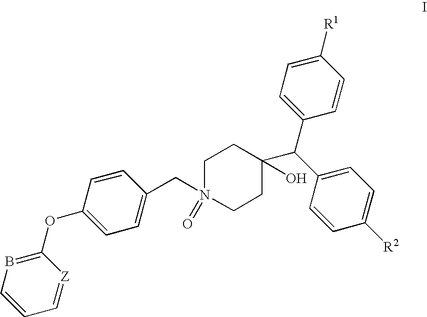
wherein
R¹ and R² are independently selected from the group consisting of halogen, CF₃, OCF₃, OCHF₂, OCF₂CHF₂ and SF₅; and
Z and B are independently selected from the group consisting of CH and N;
said process comprising:
a) forming intermediate (B): 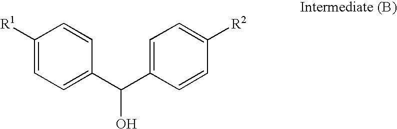
wherein R¹ and R² are as defined above;
by reacting two substituted aryl halides of formula (A-1) and formula (A-2): 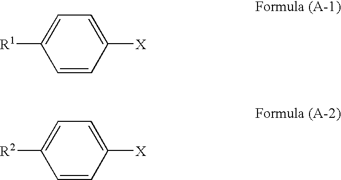
wherein
X is halogen; and
R¹ and R² are as defined above;
with a Grignard reagent and an alkyl formate;
b) halogenating intermediate (B) to form intermediate (C): 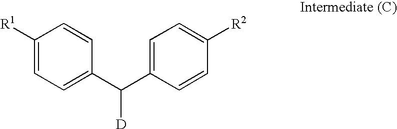
wherein
D is halogen; and
R¹ and R² are as defined above;
c) reacting intermediate (C) with a compound of formula (D): 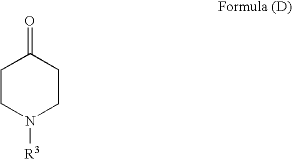
wherein R³ is selected from the group consisting of hydrogen, benzyl, substituted benzyl, t-butoxycarbonyl and trimethylsilyl;
to form intermediate (E): 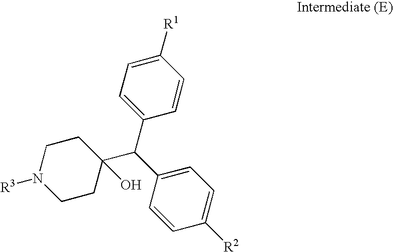
wherein R¹, R² and R³ are as defined above;
d) reacting intermediate (E) with an acid in the presence of a catalyst to form intermediate (F): 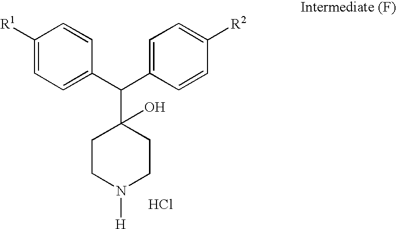
wherein R¹ and R² are as defined above;
e) reacting a substituted phenol and a compound selected from the group consisting of 2-halopyridine, 2-halopyrimidine and halobenzene to form intermediate (G): 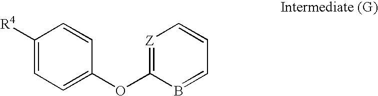
wherein
R⁴ is —CH₃ or —CHO; and
Z and B are as defined above;
f) when R⁴ in intermediate (G) is —CH₃, halogenating intermediate (G) to form intermediate (H): 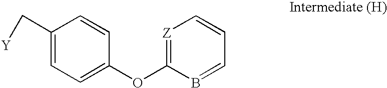
wherein
Y is halogen; and
Z and B are as defined above;
g) reacting intermediate (F) and a compound selected from the group consisting of i) intermediate (G) wherein R⁴ is —CHO and ii) intermediate (H) to form intermediate (J): 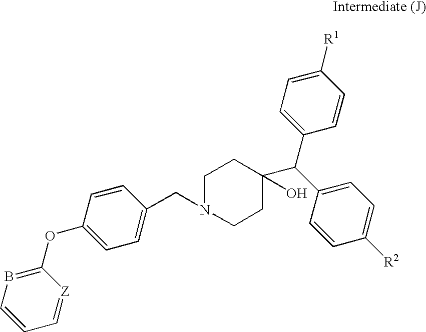
wherein R¹, R², Z and B are as defined above; and
h) oxidizing intermediate (J) to form a compound of formula I.

2. The process of claim 1 wherein R¹ and R² are OCF₃; Z is N; and B is CH.

3. The process of claim 1 wherein X and D are bromine.

4. The process of claim 1 wherein Y is bromine, iodine or chlorine.

5. The process of claim 1 wherein the alkyl formate used in step a) is ethyl formate or methyl formate.

6. The process of claim 1 wherein the Grignard reagent used in step a) is formed by reacting an alkyl magnesium halide with a substituted aryl halide.

7. The process of claim 6 wherein the alkyl magnesium halide is i-propyl magnesium chloride or i-propyl magnesium bromide.

8. The process of claim 1 wherein the reacting of step a) is conducted at a temperature in the range of from −20° C. to 30° C.

9. The process of claim 1 wherein the halogenating of step b) is conducted with hydrogen bromide and acetic acid.

10. The process of claim 1 wherein the halogenating of step b) is conducted in the presence of an organic solvent.

11. The process of claim 1 wherein the reacting of step c) is conducted at a temperature in the range of from −20° C. to −100° C.

12. The process of claim 1 wherein the reacting of step c) is conducted in the presence of an organometallic reagent.

13. The process of claim 1 wherein the acid used in step d) is formic acid.

14. The process of claim 1 wherein the catalyst used in step d) is Pd(OH)₂/C, Pd/C, Pd/SiO₂ or Pd/Al₂O₃.

15. The process of claim 1 wherein the reacting of step d) is conducted at a temperature in the range of from ambient temperature to 130° C.

16. The process of claim 1 wherein the reacting of step d) is in the presence of an alcohol solvent.

17. The process of claim 1 wherein the reacting of step e) is of a substituted phenol and 2-chloropyridine.

18. The process of claim 17 wherein the substituted phenol is 4-methylphenol or 4-hydroxybenzaldehyde.

19. The process of claim 1 wherein the reacting of step e) is conducted at a temperature in the range of from 125° C. to 180° C.

20. The process of claim 1 wherein the reacting of step e) is conducted in the presence of a base and a copper catalyst selected from the group consisting of copper, copper chloride, copper oxide, copper bromide and copper carbonate.

21. The process of claim 20 wherein the base is carbonate or hydroxide.

22. The process of claim 1 wherein the reacting of step g) when R⁴ is CHO is conducted in the presence of sodium borohydride and a solvent selected from the group consisting of 1,2-dichloroethane, dichloromethane, acetonitrile and tetrahydrofuran.

23. The process of claim 1 wherein the oxidation of step h) is conducted in the presence of an oxidizing agent and a solvent.

24. The process of claim 23 wherein the oxidizing agent is peroxide and the solvent is an alcohol solvent.

25. The process of claim 1 wherein the oxidizing of step h) is conducted at a temperature in the range of from ambient temperature to 60° C.

26. A process for preparing a compound of formula B: 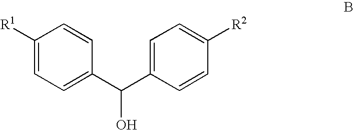
wherein R¹ and R² are independently selected from the group consisting of halogen, CF₃, OCF₃, OCHF₂, OCF₂CHF₂ and SF₅;
said process comprising reacting two substituted aryl halides of formula (A-1) and formula (A-2): 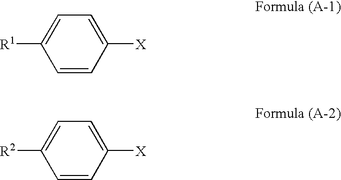
wherein
X is halogen; and
R¹ and R² are as defined above;
with a Grignard reagent and an alkyl formate.

27. A process for preparing a compound of formula E: 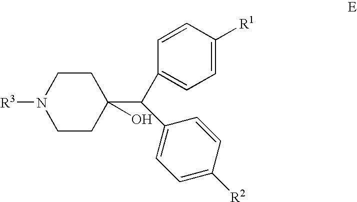
wherein
R¹ and R2 are independently selected from the group consisting of halogen, CF₃, OCF₃, OCHF₂, OCF₂CHF₂ and SF₅; and
R³ is selected from the group consisting of hydrogen, benzyl, substituted benzyl, t-butoxycarbonyl and trimethylsilyl;
said process comprising reacting a compound of formula C: 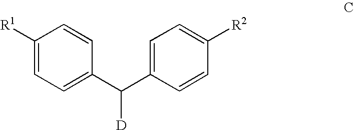
wherein
D is halogen; and
R¹ and R² are as defined above;
with a compound of formula (D): 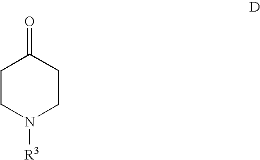
wherein R³ is as defined above.

28. A process for preparing a compound of formula J: 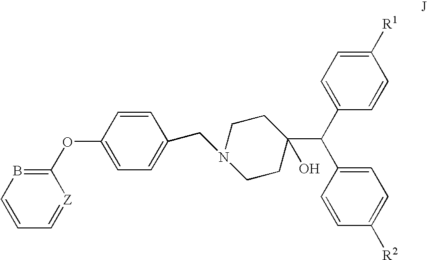
wherein
R¹ and R² are independently selected from the group consisting of halogen, CF₃, OCF₃, OCHF₂, OCF₂CHF₂ and SF₅; and
Z and B are independently selected from the group consisting of CH and N;
said process comprising reacting a compound of formula F: 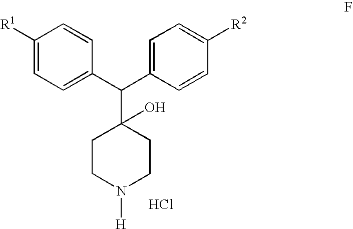
and a compound selected from the group consisting of i) a compound of formula G: 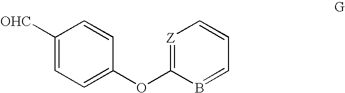
wherein Z and B are as defined above;
and ii) a compound of formula H: 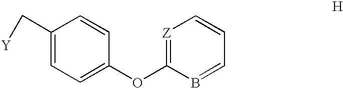
wherein
Y is halogen; and
Z and B are as defined above.

29. A compound of formula E: 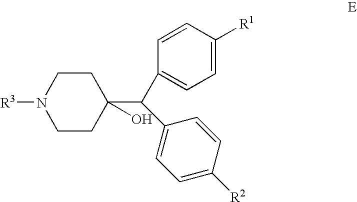
wherein
R¹ and R² are independently selected from the group consisting of halogen, CF₃, OCF₃, OCHF₂, OCF₂CHF₂ and SF₅; and
R³ is selected from the group consisting of benzyl and trimethylsilyl.
